(19) United States
(12) Patent Application Publication
(10) Pub. No.: US 2003/0219485 A1
(43) Pub. Date: Nov. 27, 2003

(54) Oral osmotic controlled drug delivery system

(75) Inventors: Nitin Bhalachandra Dharmadhikari, Nagpur (IN); Ashish Prabhakar Mungre, Mumbai (IN)

Correspondence Address: ARMSTRONG,WESTERMAN & HATTORI, LLP, 1725 K STREET, NW, SUITE 1000, WASHINGTON, DC, 20006, US

(73) Assignee: SUN PHARMACEUTICAL INDUSTRIES LIMITED, Mumbai (IN)

(21) Appl. No.: 10/437,045

(22) Filed: May 14, 2003

(30) Foreign Application Priority Data
May 15, 2002 (IN) 437/MUM/2002

(51) Int. Cl. A61K 31/426; A61K 31/734; A61K 9/24

(52) U.S. Cl. 424/473; 514/54; 514/469

(57) ABSTRACT

The present invention provides an oral osmotic controlled drug delivery system comprising-

(a) a core comprising a homogeneous mixture of glipizide, a hydrophilic polymer and other pharmaceutically acceptable excipients,

(b) a semipermeable wall surrounding the core, said wall being impermeable to the contents of the core, but permeable to fluids present in the environment of use, and

(c) a passageway through the wall for release of contents of the core to the environment of use,

wherein components of the system are used in amounts such that the oral osmotic controlled drug delivery system is bioequivalent to Glucotrol® XL, the controlled release glipizide formulation commercially available in the United States of America.

### FIELD OF THE INVENTION

The present invention relates to an oral osmotic controlled drug delivery system. More particularly, the present invention relates to an oral osmotic controlled drug delivery system for glipizide.

### BACKGROUND OF THE INVENTION

Theeuwes and Higuchi in U.S. Pat. Nos. 3,845,770 ('770) and 3,916,899 ('899) disclosed an advance in the delivery of therapeutic agents using an osmotic system comprising a semipermeable wall that surrounds a compartment containing an active agent. In this system the wall is permeable to the passage of an external fluid and substantially impermeable to the passage of the therapeutic agent. The therapeutic agent is delivered through a passageway in the wall. The surrounding external fluid is imbibed through the semipermeable wall into the compartment at a rate determined by the permeability of the semipermeable wall and the osmotic pressure gradient across the semipermeable wall. An aqueous solution containing the therapeutic agent is delivered through the passageway. The systems are effective for delivering a therapeutic agent that is soluble in the fluid and exhibits an osmotic pressure gradient across the semipermeable wall against the external fluid.

The osmotic systems disclosed in the '770 and '899 patents are unsuitable for therapeutic agents having low solubility in water because the osmotic pressure generated by such an agent on its own is too low to cause release of the agent formulation from the core at a constant rate. Theeuwes, in U.S. Pat. No. 4,111,202, disclosed an osmotic device with enhanced ability for delivering therapeutic agents, including agents that are insoluble to very soluble in the fluid. This osmotic device has a therapeutic agent compartment and an osmogent compartment, also called a “push” compartment, separated by an internal film. The fluid imbibed through the semipermeable wall into the osmogent compartment causes the compartment to increase in volume. This pushes the film against the therapeutic agent compartment thereby delivering the therapeutic agent through the passageway of the osmotic device to the external environment. This system was a pioneer system in what is generally referred to as “push-pull systems” by those skilled in the art. While this device operates successfully for its intended use, and while it can deliver numerous therapeutic agents of varying solubilities, its use can be limited because of the manufacturing steps and costs needed for fabricating and placing the movable film in the compartment of the osmotic device.

U.S. Pat. No. 4,612,008 discloses device comprising a semipermeable wall covering a compartment comprising a first composition consisting of the therapeutic agent, an osmogent and an osmopolymer, and a second composition comprising an osmogent and an osmopolymer, and a passageway in the semipermeable wall for release of the therapeutic agent. The components of the second composition swell upon intake of fluid from the external environment, and cause release of the therapeutic agent of the first composition through the passageway. Similarly, U.S. Pat. Nos. 5,082,668, 5,091,190, 5,545,413 and 5,591,454 also disclose osmotic drug delivery systems wherein the core comprises a first layer comprising the active ingredient and a second “push compartment” or a “displacement lamina”, the latter being capable of swelling and pushing or displacing the active ingredient from the core. In all of the above-mentioned systems, these layers are present as two physically separate layers within the osmotic drug delivery device. This type of push-pull system was developed to provide a glipizide controlled release formulation, which is commercially available in the United States of America under the tradename Glucotrol® XL. However, these patents do not disclose single compartment osmotic drug delivery systems wherein the core comprises a homogenous mixture of the active ingredient, a hydrophilic polymer and a superdisintegrant or a highly swellable polymer.

U.S. Pat. No. 4,327,725 ('725) provides an osmotic dispensing device for delivering therapeutic agents that are difficult to deliver in meaningful amounts because of their low solubilities in aqueous and biological fluids. The osmotic device of this patent comprises a semipermeable wall surrounding a compartment containing a therapeutic agent that is insoluble to very soluble in aqueous and biological fluids, and an expandable hydrogel. Upon uptake of external fluid, the hydrogel expands, and in some operations mixes with the therapeutic agent, thereby forming a dispensable formulation that is dispensed through the passageway of the device. This device operates successfully for its intended use, and it delivers many difficult to deliver therapeutic agents for their intended purpose. However, the applicability and suitability of this system to deliver glipizide in a manner such that the system is bioequivalent to glipizide controlled release formulation commercially available in the United States of America was not disclosed. Given the difficulty of delivering low aqueous solubility drugs, such as glipizide, with osmotic systems, and given the fact that the more complex push-pull system of U.S. Pat. No. 4,612,008 was actually used to deliver glipizide in the commercially available Glucotrol® XL, a person skilled in the art would doubt that the single compartment system could be successfully employed to provide a system that is bioequivalent to Glucotrol® XL.

U.S. Pat. No. 4,992,278 ('278) presents a problem associated with single compartment osmotic drug delivery systems and teaches that when known swelling agents such as polyvinylpyrrolidone, polyethylene oxide, polymethacrylate and the like, are used in single compartment systems the swelling pressure is so great that in contact with water the semi-permeable membrane bursts and the whole system disintegrates in the stomach after a short time. The problem was solved by the advantageous swelling polymer mixture of the '278 patent consisting of a mixture of a vinyl pyrrolidone/vinyl acetate copolymer with an ethylene oxide homopolymer. However, the patent does not disclose single compartment osmotic drug delivery systems wherein the core comprises a homogenous mixture of the active ingredient, a hydrophilic polymer and a superdisintegrant or a highly swellable polymer.

### OBJECT OF THE INVENTION

It is the object of the present invention to provide an oral osmotic controlled drug delivery system comprising a core comprising a homogenous mixture of glipizide, a hydrophilic polymer and pharmaceutically acceptable excipients, the core being surrounded by a semipermeable membrane having a passageway therein for the release of the drug.

It is another object of the present invention to provide an oral osmotic controlled drug delivery system comprising a core comprising a homogenous mixture of glipizide, a hydrophilic polymer and a superdisintegrant or a highly swellable polymer, the core being surrounded by a semipermeable membrane having a passageway therein for the release of the drug. Apparently, the objective is inclusive of achieving two features in the osmotic drug delivery system, i.e. the system be capable of delivering a low solubility drug such as glipizide at a desirable rate by development of internal pressure to push the glipizide and yet not develop excessive pressure so that the semipermeable membrane bursts.

It is also the object of the present invention to provide a simpler, easier to manufacture, single compartment oral osmotic controlled drug delivery system for glipizide, wherein the components are used such that the system is bioequivalent with commercially available controlled release formulations of glipizide.

### SUMMARY OF THE INVENTION

The present invention provides an oral osmotic controlled drug delivery system comprising-

(a) a core comprising a homogeneous mixture of glipizide, a hydrophilic polymer and other pharmaceutically acceptable excipients,

(b) a semipermeable wall surrounding the core, said wall being impermeable to the contents of the core, but permeable to fluids present in the environment of use, and

(c) a passageway through the wall for release of contents of the core to the environment of use,

wherein components of the system are used in amounts such that the oral osmotic controlled drug delivery system is bioequivalent to Glucotrol® XL, the controlled release glipizide formulation commercially available in the United States of America.

The present invention also provides an oral osmotic controlled drug delivery system comprising-

(a) a core comprising a homogeneous mixture of glipizide, a first hydrophilic polymer, and a second hydrophilic polymer selected from the group consisting of a superdisintegrant and a highly swellable polymer capable of swelling to at least twice its volume when exposed to an aqueous environment, and other pharmaceutically acceptable excipients,

(b) a semipermeable wall surrounding the core, said wall being impermeable to the contents of the core, but permeable to fluids present in the environment of use, and

(c) a passageway through the wall for release of contents of the core to the environment of use.

We have found a novel osmotic controlled drug delivery system for oral administration of glipizide, which utilizes a homogenous mixture of a hydrophilic polymer, or a mixture of hydrophilic polymer and a superdisintegrant or a highly swellable polymer, such that upon contact with water the swelling pressure generated is not so great that the semi-permeable membrane bursts, and at the same time the mixture of the hydrophilic polymer and the superdisintegrant or the highly swellable polymer have a high degree of swelling such that they are usable in small amounts.

### DETAILED DESCRIPTION OF THE PRESENT INVENTION

The present invention provides an oral osmotic controlled drug delivery system comprising-

(a) a core comprising a homogeneous mixture of glipizide, a hydrophilic polymer and other pharmaceutically acceptable excipients,

(b) a semipermeable wall surrounding the core, said wall being impermeable to the contents of the core, but permeable to fluids present in the environment of use, and

(c) a passageway through the wall for release of contents of the core to the environment of use,

wherein components of the system are used in amounts such that the oral osmotic controlled drug delivery system is bioequivalent to Glucotrol® XL, the controlled release glipizide formulation commercially available in the United States of America.

The present invention also provides an oral osmotic controlled drug delivery system comprising-

(a) a core comprising a homogeneous mixture of glipizide, a first hydrophilic polymer, and a second hydrophilic polymer selected from the group consisting of a superdisintegrant and a highly swellable polymer capable of swelling to at least twice its volume when exposed to an aqueous environment, and other pharmaceutically acceptable excipients,

(b) a semipermeable wall surrounding the core, said wall being impermeable to the contents of the core, but permeable to fluids present in the environment of use, and

(c) a passageway through the wall for release of contents of the core to the environment of use.

Glipizide, a blood-glucose lowering agent, is preferably used in amounts ranging from about 2 mg to about 15 mg in the oral osmotic controlled drug delivery system of the present invention.

The oral osmotic controlled drug delivery system of the present invention provides blood plasma levels of glipizide that are effective in the treatment of diabetes mellitus type II, while being bioequivalent with commercially available controlled release formulations of glipizide.

Suitable hydrophilic polymers that may be used in the present invention are selected from among polymers that can be of plant, animal, mineral or synthetic origin. Examples of such polymers include (A) cellulose derivatives such as hydroxy C₁₋₄ alkyl celluloses, hydroxy C₁₋₄ alkyl C₁₋₄ alkyl celluloses, carboxyalkyl celluloses and the like; (B) vinyl pyrrolidone polymers such as polyvinylpyrrolidone, crosslinked polyvinylpyrrolidone or crospovidone; (C) copolymers of vinyl pyrrolidone and vinyl acetate; (D) gums of plant, animal, mineral or synthetic origin such as (i) agar, alginates, carrageenan, furcellaran derived from marine plants, (ii) guar gum, gum arabic, gum tragacanth, karaya gum, locust bean gum, pectin derived from terrestrial plants, (iii) microbial polysaccharides such as dextran, gellan gum, rhamsan gum, welan gum, xanthan gum, and (iv) synthetic or semi-synthetic gums such as propylene glycol alginate, hydroxypropyl guar and modified starches like sodium starch glycolate. The swellable hydrophilic polymers are present in suitable amounts such that the polymeric swelling agent exhibits controlled swelling and the wall does not rupture or burst, the desired rate of drug delivery is obtained and the polymeric swelling agent does not contribute significantly to increasing the size of the osmotic system. The core of the oral osmotic controlled drug delivery system of the present invention may include one or more of the above hydrophilic polymers.

Xanthan gum is a high molecular weight microbial polysaccharide gum obtained by the aerobic fermentation of carbohydrates with Xanthomonas campestris. It may be used as the preferred hydrophilic polymer in the present invention. Xanthan gum is available in several different grades that have varying particle sizes, and is available commercially as Rhodigel, Rhodigel EZ, Rhodigel 200, Keltrol T and Xanthan gum Type FF. A preferred embodiment of the present invention contains xanthan gum having a particle size such that 100% of the particles pass through ASTM 80#, and a minimum of 92% pass through ASTM 200# (where ASTM stands for American Society for Testing and Materials, and 80# indicates a sieve with 80 meshes, each of size 180 μm, present in a length of 2.54 cm in each transverse direction parallel to the wires, and 200# indicates a sieve with 200 meshes, each of size 751 μm, present in a length of 2.54 cm in each transverse direction parallel to the wires, the sieve being made of stainless steel, brass or other inert material), and wherein the viscosity of a 1% solution of the xanthan gum in 1% KCl solution at 25° C. is 1400 cP. The xanthan gum may be present in the core of the oral osmotic controlled drug delivery system in an amount from about 1% to about 5% by weight of the core.

Vinylpyrrolidone polymers, also known as polyvinyl pyrrolidone or Povidone, are synthetic polymers consisting essentially of linear 1-vinyl-2-pyrrolidinone groups, the degree of polymerization of which results in polymers of various molecular weights, the molecular weight ranging between 2500 and 3,000,000 Daltons. PVP is commercially available as Kollidon® (BASF), Plasdone® and Peristone® (General Aniline). PVP is classified into different grades on the basis of its viscosity in aqueous solution. Different grades of PVP available are PVP K-12, PVP K-15, PVP K-17, PVP K-25, PVP K-30, PVP K-60, PVP K-90 and PVP K-120. The K-value referred to in the above nomenclature is calculated from the viscosity of the PVP in aqueous solution, relative to that of water. PVP may be used in the present invention as a hydrophilic polymer, either alone or in combination with another hydrophilic polymer. In preferred embodiments the PVP used is PVP K-30 having an approximate molecular weight of 50,000 Daltons. It is used in an amount ranging from about 0.5% to about 10% by weight of the system, more preferably from about 1% to about 5% by weight of the core.

In one embodiment of the present invention, the hydrophilic polymer used comprises a mixture of polyvinylpyrrolidone and xanthan gum.

In yet another embodiment the core of the oral osmotic controlled drug delivery system of the present invention comprises xanthan gum as the first hydrophilic polymer and a superdisintegrant as the second hydrophilic polymer.

Examples of superdisintegrants that may be used in the present invention include sodium starch glycolate, crospovidone, croscarmellose sodium, and mixtures thereof. All these superdisintegrants are insoluble in aqueous fluids and have a very high tendency to absorb fluids, thereby leading to their swelling. Sodium starch glycolate is a sodium salt of carboxymethyl ether of starch, and is commercially available as Vivastar®, Primojel and Explotab. Crospovidone is a water insoluble synthetic crosslinked homopolymer of N-vinyl-2-pyrrolidinone, commercially available as Kollidon and Polyplasdone. Croscarmellose sodium is a crosslinked polymer of sodium carboxymethyl cellulose, also known as Ac-Di-Sol, and available commercially as Nymcel® ZSX, Pharmacel® XL, Primellose® or Solutab®. Superdisintegrants are used in an amount ranging from about 0.5% to about 5% by weight of the core, preferably from about 1% to about 3% by weight of the core.

We have found that an oral osmotic controlled drug delivery system for glipizide may be obtained by using (i) a hydrophilic polymer, or (ii) a mixture of a hydrophilic polymer with a superdisintegrant or a highly swellable polymer. As used herein, the term “highly swellable polymer” includes polymers that swell to at least twice their volume upon exposure to an aqueous environment. The highly swellable polymer used in the core of the oral osmotic controlled drug delivery system of the present invention may include one or more polymers selected from the group comprising—poly(ethylene oxide), cellulose, alkyl-substituted celluloses such as hydroxymethyl cellulose, hydroxyethyl cellulose, hydroxypropyl cellulose, hydroxypropyl methylcellulose, carboxymethyl cellulose and its alkali salts, crosslinked polyacrylic acids, xanthan gum, vinyl pyrrolidone polymers, copolymers of vinyl pyrrolidone and vinyl acetate, and mixtures thereof. Polymers or grades of the above-mentioned polymers that swell to at least twice their volume when exposed to an aqueous environment are selected.

The core of the oral osmotic drug delivery system of the present invention may further include pharmaceutically acceptable excipients such as swelling agents, water-soluble compounds for inducing osmosis, wetting agents and the like.

Examples of swelling agents that may be used in the core of the oral osmotic controlled drug delivery system of the present invention include cellulose derivatives such as methyl cellulose, ethyl cellulose, hydroxypropyl cellulose, hydroxypropyl methylcellulose, carboxymethyl cellulose and its alkali salts, and the like, and mixtures thereof. The swelling agents may be used in an amount ranging from about 0.5% to about 5% by weight of the core. In one embodiment the swelling agent used is sodium carboxymethyl cellulose in an amount ranging from about 1% to about 3% by weight of the core.

The core of the oral osmotic controlled drug delivery system of the present invention may further include osmotic agents. The osmotic agents, or osmogents, that may be used in the system of the present invention include all pharmaceutically acceptable and pharmacologically inert water-soluble compounds referred to in the pharmacopoeias such as United States Pharmacopoeia, as well as in Remington: The Science and Practice of Pharmacy. Pharmaceutically acceptable water-soluble salts of inorganic or organic acids, or non-ionic organic compounds with high water solubility, e.g. carbohydrates such as sugar, or amino acids, are generally preferred. The examples of agents used for inducing osmosis include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like, and mixtures thereof. The amount of osmogents that may be used depends on the particular osmogent that is used and may range from about 1% to about 95% by weight of the core. In one embodiment the osmogent used is a mixture of sodium chloride and lactose monohydrate.

Wetting agents that may be used in the oral osmotic controlled drug delivery system of the present invention include cationic surfactants such as quarternary ammonium compounds, anionic surfactants such as sodium docusate, sodium lauryl sulfate and the like, non-ionic surfactants such as polyoxyethylene fatty acid esters (polysorbates) and sorbitan fatty acid esters. In preferred embodiments the surfactant is used in an amount ranging from about 0.1% to about 5% by weight of the core, more preferably from about 0.1% to about 1% by weight of the core. Sodium lauryl sulfate is used as the preferred surfactant.

Further, additional pharmaceutical excipients may be present in the core. Examples of other additional excipients include those excipients which are used in tabletting, during the preparation of granules, e.g. binders, lubricants, glidants, dispersants, colorants and the like. Thus, it is possible to use conventional adjuvants like lactose, saccharose, sorbitol, mannitol, cellulose, microcrystalline cellulose, or magnesium stearate, in addition to those mentioned above. The lubricants are typically present in an amount ranging from 0.5% to 5% by weight of the core. In one embodiment of the present invention sodium lauryl sulfate is included as the surfactant, in an amount ranging between 0.1% and 5% by weight of the core.

The suitable materials that may be used in the present invention for forming the semi-permeable wall include polymeric microporous materials that are well known to those skilled in the art and have been described in prior art, for example in U.S. Pat. No. 4,857,336 (RE 34990) and U.S. Pat. No. 5,284,662. The cellulose acetates are preferred materials for wall formation. A combination of cellulose acetates with different degrees of acetylation may be employed to form the semi-permeable wall. As the degree of acetylation of the cellulose acetate increases, the material becomes more impermeable to aqueous fluids. Hence, a suitable combination of the cellulose acetates should be used to impart impermeability to the wall. A hydroxy C₁-C₄ alkyl C₁-C₄ alkyl cellulose and a plasticiser may also be present as components of the semi-permeable wall.

A preferred combination for forming the wall is cellulose acetate, of two different types, with different degrees of acetylation, in an amount ranging from about 78% to about 82% by weight of the wall, a hydroxy C₁-C₄alkyl-C₁-C₄alkyl cellulose, preferably hydroxypropyl methylcellulose, present in an amount ranging between about 5% to about 10% by weight of the wall, a poly C₂-C₄ alkylene glycol, preferably polyethyleneglycol, more preferably, polyethyleneglycol 8000, in an amount ranging from about 10% to about 14% by weight of the wall, and a suitable solvent system to form the coating solution. A preferred embodiment of the invention contains cellulose acetate 320S and cellulose acetate 398-10 NF, with the weight ratio of 320 S:398-10 NF being about 5:1 to about 8:1.

The expression “a passageway through the wall for release of contents of the core to the environment of use” covers a suitable means for releasing the drug formulation from the delivery system. This passageway comprises of orifices, bores or apertures and the like, through the semi-permeable wall prepared by various methods such as those mentioned in U.S. Pat. No. 3,916,899. The passageway acts as the connection between the drug-containing core and the aqueous fluid in the environment. The most suitable form of passageway is an orifice formed by mechanical or laser drilling of the semi-permeable wall.

The oral osmotic controlled drug delivery system of the present invention is prepared by known methods, e.g. mixing, granulation, compression, coating etc. The mixture can be dry granulated, wet granulated or can be directly compressed. In the wet granulation process, the drug is mixed with the hydrophilic polymer and the other excipients to obtain a dry blend. It is then granulated using a granulating agent. Water is the preferred granulating agent. The granules, after lubrication, are eventually compressed on a rotary compression machine using standard punches. In case of dry granulation, the dry mixture of the drug, the hydrophilic polymer and other excipients is passed through a chilsonator to obtain slugs of the material, which are then passed through suitable sieves to obtain granules. These granules are lubricated with a suitable lubricant and compressed on a rotary compression machine. In case of direct compression, the components of the system are mixed thoroughly and directly compressed on a rotary compression machine. The compressed cores, obtained by any one of the above methods, are subjected to coating, moulding, spraying, or immersion in a solution of a suitable material, to form the semi-permeable wall. An orifice is finally drilled into the semi-permeable wall using mechanical or laser drilling. The oral osmotic system thus obtained may optionally be coated with a non-functional film coat by conventional methods known to a person skilled in the art.

The present invention provides an oral osmotic controlled drug delivery system that releases glipizide in a controlled manner to provide desirable blood level profile of glipizide that provides efficacy in the treatment of diabetes. For example, when administered as a single dose in fasted state to healthy human subjects it provides area under the plasma concentration-time curve (AUC) which is comparable to that provided by the oral controlled drug delivery system commercially available in the United States of America. Alternatively, it provides peak plasma levels (Cₘₐₓ) that are comparable with those provided by the oral controlled drug delivery system commercially available in the United States of America. Herein, the term capable means that 90 percent confidence intervals for the ratio of the population geometric means between the oral osmotic controlled drug delivery system of the present invention and the oral controlled drug delivery system commercially available in the United States of America, namely Glucotrol XL®, based on log-transformed data, is contained in the limits of 70-135 percent for AUC and Cₘₐₓ. More preferred embodiments of the present invention are bioequivalent to marketed glipizide controlled drug delivery systems. Bioequivalence may be determined according to United States Food and Drug Administration (USFDA) guidelines and criteria.

The examples that follow do not limit the scope of the present invention and are merely used as illustrations.

### EXAMPLE 1

The oral osmotic controlled drug delivery system of the present invention was obtained as per the formula given in Table 1 below-

TABLE 1

| | Quantity | Percent (%) by |
|---|---|---|
| Ingredients | (mg/tab) | weight of the core |
| Core | | |
| Glipizide | 5.5 | 3.33 |
| Sodium starch glycolate | 2.5 | 1.52 |
| Xanthan gum | 10.0 | 6.06 |
| Sodium chloride | 15.0 | 9.09 |
| Lactose monohydrate | 130.35 | 79.0 |
| Magnesium stearate | 1.65 | 1.00 |
| Coat | | |
| Cellulose acetate 320S | 1.077 | Coated to a weight |
| Cellulose acetate 398 10 | 0.247 | gain of 20% w/w of |
| Hydroxypropyl methylcellulose | 0.126 | the core |
| E15 | | |
| Polyethylene glycol 8000 | 0.198 | |

Glipizide, sodium starch glycolate, xanthan gum, lactose monohydrate were passed through ASTM (American Society for Testing and Materials) #40 sieve. Sodium chloride was passed through ASTM #60 sieve. All the ingredients were mixed to obtain a dry blend and granulated using water as the granulating agent. The granules obtained were dried in a fluid bed dryer to a moisture content of 2%, and passed through ASTM #20. The granules were then lubricated with magnesium stearate, previously passed through ASTM #40 sieve. The lubricated mass was compressed on a rotary compression machine to obtain the core, which was coated with the coating solution in a Glatt Wurster coater to a defined weight gain. The tablets were then dried in a tray dryer for 48 hours. A passageway was made by drilling an orifice on one side of the coated tablet, by manual or laser drilling.

### EXAMPLE 2

The oral osmotic controlled drug delivery system of the present invention was obtained as per Table 2 below-

TABLE 2

| | Quantity | Percent (%) by |
|---|---|---|
| Ingredients | (mg/tab) | weight of the core |
| Core | | |
| Glipizide | 5.5 | 3.33 |
| Sodium carboxymethyl | 2.5 | 1.52 |
| cellulose | | |
| Xanthan gum | 2.0 | 1.21 |
| Sodium chloride | 15.0 | 9.09 |
| Sodium lauryl sulfate | 0.42 | 0.25 |
| Polyvinylpyrrolidone (PVP K- | 5.00 | 3.03 |
| 30) | | |
| Lactose monohydrate | 132.93 | 80.56 |
| Magnesium stearate | 1.65 | 1.00 |
| Coat | | |
| Cellulose acetate 320S | 2.4096 | Coated to a weight |
| Cellulose acetate 398 10 | 1.6064 | gain of 18-20% w/w |
| Hydroxypropyl methylcellu- | 0.382 | of the core |
| lose (HPMC E15) | | |
| Polyethylene glycol 8000 | 0.602 | |
| Non-functional coat | | |
| Opadry white/blue | Used as a | Coated to a weight |
| | 15-17% aqueous | gain of 5% w/w of |
| | solution | the core |

Glipizide, sodium carboxymethyl cellulose, xanthan gum, sodium chloride, PVP K-30 and lactose monohydrate were passed through suitable ASTM sieves and mixed in a mixer to obtain a dry blend. This blend was granulated using water. The granules thus obtained in a fluid bed dryer to moisture content of about 2%, and sifted through ASTM #20. The granules were then lubricated with magnesium stearate previously passed through ASTM #40, and compressed on a rotary compression machine to a target weight of 165 mg. The compressed cores thus obtained were coated to a weight gain of 18-20% w/w of the core, with a coating solution comprising the cellulose acetates, HPMC and PEG 8000 in a mixture of dichloromethane and methanol. The coated tablets were dried in a tray dryer at about 40° C. for 12-24 hours. A passageway was made by drilling an orifice on one side of the coated tablet, by manual or laser drilling. The drilled tablets were finally coated with the non-functional coat to a weight gain of 5% w/w.

The tablets thus obtained and commercially available glipizide controlled release tablets, Glucotrol® XL (5 mg), were subjected to dissolution test using United States Pharmacopoeia dissolution apparatus, type II. The dissolution medium used was 900 ml of phosphate buffer pH 6.8, at 37±0.5° C., at a speed of 100 rpm. The results of the dissulotion are recorded in Table 3 below.

TABLE 3

| | Percent drug released | |
|---|---|---|
| | | Oral osmotic controlled |
| | Glucotrol ® | release tablets of Example |
| Time (hours) | XL (5 mg) | 2 |
| 2 | 2 | 3 |
| 4 | 16 | 23 |
| 6 | 32 | 51 |
| 8 | 50 | 66 |
| 10 | 69 | 77 |
| 12 | 86 | 85 |
| 16 | 108 | 91 |
| 20 | 113 | 94 |
| 24 | 114 | 96 |

### EXAMPLE 3

The bioavailability of the oral controlled drug delivery system for glipizide (Example 2) and that of marketed glipizide controlled drug delivery systems was studied. A single dose, open label, randomized, comparative, two-way crossover study was carried out for the same. Glucotrol® XL 5 mg tablets (Pfizer, USA, Lot No. 0447K01A) were used as the reference standard.

The pharmacokinetic assessment was based on the plasma levels of glipizide measured by blood sampling. Blood samples were obtained before dosing and at the following times after administration of both the reference and test medications—1, 2, 3, 4, 5, 6, 7, 8, 9, 10, 11, 12, 14, 16, 24, 28, 32 and 36 hours.

Fourteen healthy male volunteers were enrolled for the study and all of them completed the two-way crossover study. The subjects were fasted overnight before dosing and for 4 hours thereafter. Drinking water was prohibited 2 hours before dosing and 2 hours thereafter. 60 ml of 20% glucose was given at 1 and 2 hours, at every 15 minutes from 2 to 4 hours, and then at 5, 6, 7 hours after the dose. Standard meals were provided at 4, 6 and 12 hours after dosing and at appropriate times thereafter. Meal plans were identical for both the periods.

Subjects received a single tablet of glipizide (5 mg, Example 2) with 240 ml of water at ambient temperature after the overnight fast, as the test medication, while a single tablet of Glucotrol® XL 5 mg (Pfizer) was administered as the reference medication.

The plasma concentration of glipizide was determined for samples collected at different time points and averaged over the fourteen volunteers. The data is given in Table 4 below.

TABLE 4

| | Plasma concentration (ng/ml) (Mean ± SD) | |
|---|---|---|
| Time | Glipizide tablet | Glucotrol ® |
| (hrs) | (5 mg tablet, Example 2) | XL 5 mg (Pfizer) |
| 0 | 0.00 | 0.00 |
| 1 | 0.00 | 0.00 |
| 2 | 0.00 | 0.49 |
| 3 | 3.91 | 2.19 |
| 4 | 14.05 | 14.04 |
| 5 | 56.85 | 47.10 |
| 6 | 96.44 | 75.74 |
| 7 | 121.69 | 99.84 |
| 8 | 125.49 | 113.74 |
| 9 | 117.61 | 112.15 |
| 10 | 122.69 | 134.34 |
| 11 | 115.70 | 135.86 |
| 12 | 104.31 | 127.72 |
| 14 | 102.81 | 117.66 |
| 16 | 79.99 | 98.09 |
| 24 | 52.10 | 63.49 |
| 28 | 41.08 | 45.30 |
| 32 | 30.15 | 33.35 |
| 36 | 21.31 | 24.56 |
| 48 | 7.25 | 5.94 |

1. An oral osmotic controlled drug delivery system comprising-
(a) a core comprising a homogeneous mixture of glipizide, a hydrophilic polymer and other pharmaceutically acceptable excipients,
(b) a semipermeable wall surrounding the core, said wall being impermeable to the contents of the core, but permeable to fluids present in the environment of use, and
(c) a passageway through the wall for release of contents of the core to the environment of use,
wherein components of the system are used in amounts such that the oral osmotic controlled drug delivery system is bioequivalent to Glucotrol® XL, the controlled release glipizide formulation commercially available in the United States of America.

2. An oral osmotic controlled drug delivery system as claimed in claim 1, wherein the glipizide is present in an amount ranging from about 2 mg to about 15 mg.

3. An oral osmotic controlled drug delivery system as claimed in 1, wherein the hydrophilic polymer is selected from a group comprising cellulose derivatives, vinylpyrrolidone polymers, copolymers of vinylpyrrolidone and vinyl acetate, gums of plant, animal, mineral or synthetic origin, modified starches, and mixtures thereof.

4. An oral osmotic controlled drug delivery system as claimed in claim 3, wherein the hydrophilic polymer is xanthan gum.

5. An oral osmotic controlled drug delivery system as claimed in claim 4, wherein the xanthan gum used has a particle size such that about 100% of the particles pass through a sieve of ASTM 80# and a minimum of about 92% of the particles pass through a sieve of ASTM 200#.

6. An oral osmotic controlled drug delivery system as claimed in claim 5, wherein the viscosity of a 1% solution of the xanthan gum in 1% KCl solution at 25° C. is 1400 cP.

7. An oral osmotic controlled drug delivery system as claimed in claim 5, wherein the xanthan gum is used in an amount ranging from about 1% to about 5% by weight of the core.

8. An oral osmotic controlled drug delivery system as claimed in claim 3, wherein the hydrophilic polymer is polyvinylpyrrolidone.

9. An oral osmotic controlled drug delivery system as claimed in claim 8, wherein the polyvinylpyrrolidone is used in an amount ranging from about 1% to about 5% by weight of the core.

10. An oral osmotic controlled drug delivery system as claimed in claim 8, wherein the polyvinylpyrrolidone used has an approximate molecular weight of 50,000 Daltons.

11. An oral osmotic controlled drug delivery system as claimed in claim 3, wherein the hydrophilic polymer is a mixture of xanthan gum and polyvinylpyrrolidone.

12. An oral osmotic controlled drug delivery system as claimed in claim 1, wherein the core further comprises a superdisintegrant.

13. An oral osmotic controlled drug delivery system as claimed in claim 12, wherein the superdisintegrant is selected from a group comprising sodium starch glycolate, crospovidone croscarmellose sodium and mixtures thereof.

14. An oral osmotic controlled drug delivery system as claimed in claim 12, wherein the superdisintegrant is used in an amount ranging from about 0.5% to about 5% by weight of the core.

15. An oral osmotic controlled drug delivery system as claimed in claim 1 wherein the core further comprises a swelling agent.

16. An oral osmotic controlled drug delivery system as claimed in claim 15, wherein the swelling agent is selected from a group comprising cellulose derivatives such as methyl cellulose, ethyl cellulose, hydroxypropyl cellulose, hydroxypropyl methylcellulose, carboxymethyl cellulose and its alkali salts, and mixtures thereof.

17. An oral osmotic controlled drug delivery system as claimed in claim 16, wherein the swelling agent is used in an amount ranging from about 0.5% to about 5% by weight of the core.

18. An oral osmotic controlled drug delivery system as claimed in claim 16, wherein the swelling agent is sodium carboxymethyl cellulose.

19. An oral osmotic controlled drug delivery system as claimed in claim 1, wherein the core further comprises osmotic agents.

20. An oral osmotic controlled drug delivery system as claimed in claim 19, wherein a mixture of sodium chloride and lactose monohydrate is used as the osmotic agent in an amount ranging from about 15% to about 95% by weight of the core.

21. An oral osmotic controlled drug delivery system as claimed in claim 1, wherein the core further comprises a surfactant.

22. An oral osmotic controlled drug delivery system as claimed in claim 21, wherein the surfactant is used in an amount ranging from about 0.1% to about 1% by weight of the core.

23. An oral osmotic controlled drug delivery system as claimed in claim 1, wherein the semipermeable wall is made of one or more grades of cellulose acetates, hydroxypropyl methylcellulose (HPMC) and polyethylene glycol (PEG) 8000.

24. An oral osmotic controlled drug delivery system as claimed in claim 23, wherein one or more of the cellulose acetates comprises from about 78% to about 82% by weight of the wall, the HPMC comprises from about 5% to about 10% by weight of the wall, and the PEG 8000 comprises from about 10% to about 14% by weight of the wall.

25. An oral osmotic controlled drug delivery system comprising-
(a) a core comprising a homogeneous mixture of glipizide, a first hydrophilic polymer, and a second hydrophilic polymer selected from the group consisting of a superdisintegrant and a highly swellable polymer capable of swelling to at least twice its volume when exposed to an aqueous environment, and other pharmaceutically acceptable excipients,
(b) a semipermeable wall surrounding the core, said wall being impermeable to the contents of the core, but permeable to fluids present in the environment of use, and
(c) a passageway through the wall for release of contents of the core to the environment of use.

26. An oral osmotic controlled drug delivery system as claimed in claim 25, wherein the glipizide is present in an amount ranging from about 2 mg to about 15 mg.

27. An oral osmotic controlled drug delivery system as claimed in 25, wherein the first hydrophilic polymer is selected from a group comprising cellulose derivatives, vinylpyrrolidone polymers, copolymers of vinylpyrrolidone and vinyl acetate, gums of plant, animal, mineral or synthetic origin, modified starches, and mixtures thereof.

28. An oral osmotic controlled drug delivery system as claimed in claim 27, wherein the first hydrophilic polymer is xanthan gum.

29. An oral osmotic controlled drug delivery system as claimed in claim 28, wherein the xanthan gum used has a particle size such that about 100% of the particles pass through a sieve of ASTM 80# and a minimum of about 92% of the particles pass through a sieve of ASTM 200#.

30. An oral osmotic controlled drug delivery system as claimed in claim 29, wherein the viscosity of a 1% solution of the xanthan gum in 1% KCl solution at 25° C. is 1400 cP.

31. An oral osmotic controlled drug delivery system as claimed in claim 29, wherein the xanthan gum is used in an amount ranging from about 1% to about 5% by weight of the core.

32. An oral osmotic controlled drug delivery system as claimed in claim 27, wherein the first hydrophilic polymer is polyvinylpyrrolidone.

33. An oral osmotic controlled drug delivery system as claimed in claim 32, wherein the polyvinylpyrrolidone is used in an amount ranging from about 1% to about 5% by weight of the core.

34. An oral osmotic controlled drug delivery system as claimed in claim 32, wherein the polyvinylpyrrolidone used has an approximate molecular weight of 50,000 Daltons.

35. An oral osmotic controlled drug delivery system as claimed in claim 27, wherein the first hydrophilic polymer is a mixture of xanthan gum and polyvinylpyrrolidone.

36. An oral osmotic controlled drug delivery system as claimed in claim 25, wherein the first hydrophilic polymer is xanthan gum and the second hydrophilic polymer is a superdisintegrant.

37. An oral osmotic controlled drug delivery system as claimed in claim 36, wherein the superdisintegrant is selected from a group comprising sodium starch glycolate, crospovidone croscarmellose sodium and mixtures thereof.

38. An oral osmotic controlled drug delivery system as claimed in claim 36, wherein the superdisintegrant is used in an amount ranging from about 0.5% to about 5% by weight of the core.

39. An oral osmotic controlled drug delivery system as claimed in claim 25 wherein the core further comprises a swelling agent.

40. An oral osmotic controlled drug delivery system as claimed in claim 39, wherein the swelling agent is selected from a group comprising cellulose derivatives such as methyl cellulose, ethyl cellulose, hydroxypropyl cellulose, hydroxypropyl methylcellulose, carboxymethyl cellulose and its alkali salts, and mixtures thereof.

41. An oral osmotic controlled drug delivery system as claimed in claim 40, wherein the swelling agent is used in an amount ranging from about 0.5% to about 5% by weight of the core.

42. An oral osmotic controlled drug delivery system as claimed in claim 40, wherein the swelling agent is sodium carboxymethyl cellulose.

43. An oral osmotic controlled drug delivery system as claimed in claim 25, wherein the core further comprises osmotic agents.

44. An oral osmotic controlled drug delivery system as claimed in claim 43, wherein a mixture of sodium chloride and lactose monohydrate is used as the osmotic agent in an amount ranging from about 15% to about 95% by weight of the core.

45. An oral osmotic controlled drug delivery system as claimed in claim 25, wherein the core further comprises a surfactant.

46. An oral osmotic controlled drug delivery system as claimed in claim 45, wherein the surfactant is used in an amount ranging from about 0.1% to about 1% by weight of the core.

47. An oral osmotic controlled drug delivery system as claimed in claim 25, wherein the semipermeable wall is made of one or more grades of cellulose acetates, hydroxypropyl methylcellulose (HPMC) and polyethylene glycol (PEG) 8000.

48. An oral osmotic controlled drug delivery system as claimed in claim 47, wherein one or more of the cellulose acetates comprises from about 78% to about 82% by weight of the wall, the HPMC comprises from about 5% to about 10% by weight of the wall, and the PEG 8000 comprises from about 10% to about 14% by weight of the wall.
